(19) United States
(12) Patent Application Publication
(10) Pub. No.: US 2015/0297591 A1
(43) Pub. Date: Oct. 22, 2015

(54) METHODS OF TREATING PULMONARY FIBROSIS

(71) Applicant: Duke University, Durham, NC (US)

(72) Inventors: Mary E. Sunday, Durham, NC (US); Zeljko Vujaskovic, Chapel Hill, NC (US); Shutang Zhou, Chapel Hill, NC (US); W. Michael Foster, Durham, NC (US)

(21) Appl. No.: 14/410,384

(22) PCT Filed: Sep. 19, 2013
(86) PCT No.: PCT/US13/60553

§ 371 Date: Dec. 22, 2014

Related U.S. Application Data
(60) Provisional application No. 61/703,453, filed on Sep. 20, 2012.

(51) Int. Cl. A61K 31/505; G01N 33/68; C07K 16/18

(52) U.S. Cl. CPC A61K 31/505; C07K 16/18; G01N 33/6893; G01N 2333/4706

(57) ABSTRACT

A method of treating ionizing radiation injury, or pulmonary fibrosis, or pulmonary hypertension due to any cause, such as idiopathic, autoimmune, or environmental, in a mammalian subject in need thereof, comprises administering the subject a gastrin-releasing peptide (GRP) blocking agent or inhibitor in a treatment effective amount.

### RELATED APPLICATIONS

This application claims the benefit of U.S. Provisional Application No. 61/703,453, filed Sep. 20, 2012, the disclosure of which is incorporated herein in its entirety.

This application is related to U.S. patent application Ser. No. 13/201,501, previously published as PCT Application WO 2010/101990.

### GOVERNMENT SUPPORT

This invention was made with Government Support under grant nos. U19-A1067798, A1081672 and ES016347 from the National Institutes of Health. The US Government has certain rights to this invention.

### FIELD OF THE INVENTION

The present invention concerns methods for early diagnosis and treatment to prevent radiation-induced lung injury, including acute inflammation (1-4 months later), and chronic fibrosis (4-12 months later) caused by ionizing radiation (RT). The mouse model of RT-induced pulmonary fibrosis is also the best animal model for pulmonary fibrosis in humans, especially idiopathic pulmonary fibrosis (IPF), having a protracted course that does not resolve spontaneously.

### BACKGROUND OF THE INVENTION

Radiation pneumonitis (RTP), also known as “Radiation-Induced Lung Injury (RILI)”, is a serious complication affecting normal lung within a radiation field, either due to accidental exposure or due to therapeutic treatment with ionizing radiation (RT) of thoracic tumors. Despite advances in radiobiology, precise mechanisms by which RT induces lung injury remain controversial¹. Classically, RTP is characterized by a latent period that can last for months after RT exposure, followed by two stages of overt lung injury that can lead to life threatening and debilitating pulmonary toxicity^{2,3}. Acute inflammatory lung injury arises 1-6 months after RT exposure, with diffuse alveolar damage, similar to acute respiratory distress syndrome (ARDS). Later, chronic interstitial and intra-alveolar fibrosis develops, predominantly in irradiated segments, with myofibroblast differentiation and proliferation, and collagen deposition. Resolution is unusual. It is unclear why only ˜15% of RT-exposed patients develop RTP^{1,4}. General cytoprotective agents such as a catalytic antioxidant metalloporphyrin (AEOL10113) can reduce the severity of RTP by decreasing free radical injury following RT⁵.

### SUMMARY OF THE INVENTION

We propose that GRP levels at baseline (before RT) compared to GRP levels post-RT can predict which patients are at highest risk for developing RTP, including the “acute” inflammatory phase 1-4 months post-RT and the “chronic” fibrotic phase 4-12 months post-RT.

In view of the foregoing, an aspect of the present invention is a method of treating pulmonary fibrosis induced by RT or by other environmental factors, or due to altered immunity, or IPF in a mammalian subject in need thereof, comprising administering the subject a gastrin-releasing peptide (GRP) blocking agent or inhibitor in a treatment effective amount.

More generally, the present invention provides a method of treating ionizing radiation injury, or pulmonary fibrosis, or pulmonary hypertension due to any cause, such as idiopathic, autoimmune, or environmental, in a mammalian subject in need thereof, comprising administering said subject a gastrin-releasing peptide (GRP) blocking agent or inhibitor in a treatment effective amount.

In some embodiments, the radiation injury is to skin, lung, peripheral nerve, brain, or gastrointestinal tissue.

In some embodiments, wherein the radiation injury is radiation pneumonitis or radiation-induced lung injury (RTP), radiation-induced enteritis, or acute radiation syndrome.

In some embodiments, the administering step is carried out by topically applying the GRP inhibitor to airway surfaces of the subject, such as by using surfactant as a vehicle.

In some embodiments, the administering step is carried out by inhalation administration.

In some embodiments, the GRP inhibitor is a small molecule GRP inhibitor (e.g., a non-peptide GRP inhibitor); in other embodiments, the GRP inhibitor is an antibody. GRP inhibition can also be carried out by deleting or blocking either one of the two GRP receptors: GRPR and NMBR.

In some embodiments, the subject has an elevated level of GRP in at least one tissue or organ at risk of the radiation injury.

In some embodiments, the tissue or organ is lung.

In some embodiments, the method further comprises the steps of: determining an elevated level of GRP in a biological sample collected from the subject as compared to a baseline sample from the same patient prior to RT, or as compared to normal subjects; and then administering the GRP inhibitor to the subject when the elevated level is determined.

In some embodiments, the subject is a smoker or is known to have pre-existing lung disease such as chronic obstructive lung disease (COPD).

In some embodiments, the ionizing radiation injury is an acute ionizing radiation injury e.g., caused by acute exposure to ionizing radiation over a time of less than 1 or 2 days or several days more.

In some embodiments, the administering step is carried out within 1, 2, 3, or more days of the ionizing radiation injury.

In some embodiments, the administering step is carried out as a single dose or single administration of the treatment-effective amount.

In some embodiments, the administering step is carried out by parenteral injection.

A further aspect of the invention is a method of determining if a subject is at increased risk of radiation injury, by determining an elevated level of gastrin-releasing peptide (GRP) in a biological sample collected from the subject as compared to the patient's own baseline, or compared to normal subjects, such as urine; and then classifying the subject as at increased risk of radiation injury if an elevated level of GRP is determined. In some embodiments, the GRP level determining step further comprises at least partially purifying the biological sample. In some embodiments, the GRP determining step comprises an immunological, chromatographic or electrophoretic determining step.

A further aspect of the invention is the use of a GRP blocking agent to mitigate or prevent acute RTP inflammation and/or chronic pulmonary fibrosis, as described herein above or below.

A further aspect of the invention is the use of a GRP inhibitor for the preparation of a medicament for carrying out a method as described herein above or below.

A further aspect of the invention is the use of a GRP blocking agent to mitigate or prevent acute and/or chronic radiation-induced lung injury (RTP) as described herein above or below.

A further aspect of the invention is the use of a GRP blocking agent to empirically treat a patient with pulmonary fibrosis, with a treatment goal of reducing, arresting, or reversing pulmonary fibrosis of diverse causes, including idiopathic pulmonary fibrosis (IPF) as described herein above or below.

A further aspect of the invention is the use of a GRP blocking agent to empirically treat a patient at risk for pulmonary fibrosis, such as with a family history of IPF, early evidence of pulmonary fibrosis on radiographs, or following exposure to RT or drugs associated with pulmonary fibrosis such bleomycin, with a treatment goal of preventing pulmonary fibrosis of diverse causes as described herein above or below.

The foregoing and other objects and aspects of the invention are set forth in the drawings herein and the specification below. The disclosures of all US Patent references cited herein are to be incorporated herein by reference.

### BRIEF DESCRIPTION OF THE DRAWINGS

FIG. 1: Myofibroblasts (αSMA+) (A-C) & collagen staining (Masson's trichrome) (D-F) in C57BL/6 lungs at 20 wks post-15Gy±77427 given twice weekly for 20 weeks. A,D: Sham controls; B,E: 15 Gy RT+PBS; C,F: 15 Gy RT+77427.

FIG. 2: Quantitative image analysis of myofibroblasts (SMA+) & collagen (trichrome+) in C57BL/6 alveoli at 20 wks post-15Gy±77427 given IP twice weekly for 20 weeks * P<0.0001 compared to Sham+PBS; †P<0.001 compared to RT+PB.

FIG. 3: Myofibroblasts (SMA+) in C57L/J lung at 15 wks post-15Gy±77427 given as a single dose IP 24 h post-RT.

FIG. 3A: Sham: Normal SMA in smooth muscle of airways (L=lumen) and airway blood vessels (v). SMA is absent in small distal blood vessels (arrows pointing left).

FIG. 3B: RT-induces SMA+ cells around small distal blood vessels (arrows pointing right).

FIG. 3C: 77427 greatly reduces SMA+ myofibroblasts in small distal blood vessels (arrows pointing left). Note (*) shows pleural surface in all panels of FIG. 3.

FIG. 4: Quantification of the SMA immunopositive area as shown in FIG. 3 (excluding conducting airways and associated vasculature), normalized for the total area of alveolar tissue. Note: Number of C57L/J mice used for FIGS. 3 to 8: Sham n=2, 10 wkRT+PBS n=2, 10 wkRT+77427 n=2, 15 wkRT+PBS n=7, 15 wkRT+77427 n=7.

FIG. 5: Collagen staining (trichrome) of C57L/J lung at 15 wks post-15Gy±77427 given as a single dose 24 h post-RT.

FIG. 6: Quantification of trichrome staining for collagen in alveoli.

FIG. 7: Total lung collagen as determined by hydroxyproline content.

FIG. 8: Increased subpleural mast cells (toluidine blue staining) 15 weeks post-RT are reduced by single dose 77427 24 h post-RT.

### DETAILED DESCRIPTION OF ILLUSTRATIVE EMBODIMENTS

The present invention is primarily concerned with the diagnosis and treatment of human subjects, but the invention may also be carried out on animal subjects, particularly mammalian subjects such as dogs, cats, livestock and horses for veterinary purposes. Subjects may be male or female and may be of any age, including neonate, infant, juvenile, adolescent, adult, or geriatric subjects.

“Elevated level of gastrin-releasing peptide” as used refers to an amount of GRP greater than that seen in a normal subject (that is, a subject of the same species, gender, condition and age who is not at elevated risk of injury from ionizing radiation). In general, this represents about a two-fold increase in mean GRP in the urine in premature infants with chronic lung disease (bronchopulmonary dysplasia, BPD) as compared to premature infants of the same age who do not develop BPD. Urine levels of GRP have been shown to directly correlate with GRP levels in bronchoalveolar lavage (BAL) fluid²⁰, consistent with the major source of GRP being the lung¹⁶.

“Ionizing radiation” as used herein includes both electromagnetic radiation (such as X-ray radiation and gamma radiation) and particle radiation (including alpha, beta, neutron, and proton radiation). Ionizing radiation is characterized by carrying sufficient energy to ionize atoms and molecules: That is, to generate positive or negative particles from electrical neutral atoms and molecules. When passing through matter, for instance a cell, tissue, or organism, the ionizing radiation discharges energy. When sufficiently high, this can lead to acute or chronic injury to the cell, tissue or organism. See, e.g., U.S. Pat. Nos. 8,124,935; 7,649,013; and 5,663,202.

“Treat” as used herein refers to any type of treatment that imparts a benefit to a patient, including delaying the onset and/or reducing the severity of at least one symptom of the disorder (for example, decreasing cell death, and/or treating one or more of leukopenia, neutropenia, monocytopenia, lymphocytopenia, fatigue, etc.).

“Pharmaceutically acceptable” as used herein means that the compound or composition is suitable for administration to a subject to achieve the treatments described herein, without unduly deleterious side effects in light of the severity of the disease and necessity of the treatment.

“Pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299 Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

“Administering” as used herein may be by any suitable route of administration, including but not limited to topical, oral, parenteral (e.g., subcutaneous, intraveneous, intramuscular, and intraperitoneal injection, etc.), and by inhalation administration (e.g., topical application to airway surfaces).

“Antibody” as used herein refers to all types of immunoglobulins, including IgG, IgM, IgA, IgD, and IgE. Of these, IgM and IgG are particularly preferred. The antibodies may be monoclonal or polyclonal and may be of any species of origin, including (for example) mouse, rat, rabbit, horse, or human, or may be chimeric antibodies. See, e.g., M. Walker et al., Molec. Immunol. 26, 403-11 (1989). The antibodies may be recombinant monoclonal antibodies produced according to the methods disclosed in Reading U.S. Pat. No. 4,474,893, or Cabilly et al., U.S. Pat. No. 4,816,567. The antibodies may also be chemically constructed by specific antibodies made according to the method disclosed in SegAl et al., U.S. Pat. No. 4,676,980.

### 1. Active Compounds.

Active compounds useful for carrying out the present invention include peptide gastrin-releasing peptide (GRP) inhibitors and non-peptide or small molecule GRP inhibitors. Numerous such compounds are known. Examples include, but are not limited to, those described in R. Jensen et al., Pharmacological Reviews, 60, 1-42 (2008) and U.S. Pat. Nos. 5,047,502; 5,019,647; 5,109,115; 5,244,883; 5,460,801; 5,620,955; 5,620,959; 5,834,433; 6,194,437; 6,989,371; 7,147,838; and US Patent Application Publication No. 2008/0090758 (published Apr. 17, 2008). The disclosures of all United States patent references cited herein are incorporated by reference herein in their entirety.

Active compounds useful for carrying out the present invention include but are not limited to the small molecule GRP inhibitors described in F. Cuttitta et al., US Patent Application Publication No. 2008/0249115 (published Oct. 9, 2008). Particular examples of compounds described therein, and which may be used to carry out the present invention, include but are not limited to compounds of the formula:

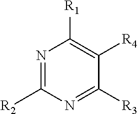

wherein:

R₁ is —R₅—(CH₂)ₙ—CH(R₆)OH, and R₅ is NH, S or O, R₆ is H or CH₃, and n is an integer from 1-4;

R₂ is NH₂, substituted amino or acetamide;

R₃ is H, halogen, CH₃, or CF₃; and

R₄ is H, alkyl, substituted alkyl, alkenyl, alkoxy or halogen; or a pharmaceutically acceptable salt or prodrug thereof. A particular example is a compound of the formula:

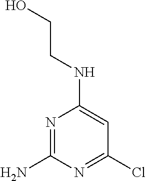 [SMILES: Nc1nc(Cl)cc(NCCO)n1]

or a pharmaceutically acceptable salt or prodrug thereof.

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

### 2. Antibody Active Compounds.

The monoclonal antibody 2A11 or monoclonal antibodies produced by the deposited cell line having the American Type Culture Collection number HB8711, are useful for carrying out the present invention, and are known and described in U.S. Pat. No. 5,109,115 to Cuttitta and Minna, the disclosure of which is incorporated herein by reference.

Antibodies that specifically bind to the epitope (or “target epitope”) bound by monoclonal antibodies produced by the deposited cell line having the ATCC No. HB8711 ((i.e., antibodies which bind to a single antigenic site or epitope on the protein) are useful for a variety of diagnostic and therapeutic purposes. Antibodies to the target epitope may be generated using methods that are well known in the art. Such antibodies may include, but are not limited to, polyclonal, monoclonal, chimeric, single chain, Fab fragments, and fragments produced by a Fab expression library. Neutralizing antibodies, (i.e., those which inhibit dimer formation) are especially preferred for therapeutic use.

Antibody fragments that contain specific binding sites for the target epitope may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (W. D. Huse et al., Science 254, 1275-1281 (1989)).

For the production of antibodies, various hosts including goats, rabbits, rats, mice, humans, and others, may be immunized by injection with the target epitope or any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

Monoclonal antibodies to the target epitope may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al, (1975) Nature 256:495-497; Kozbor, D. et al. (1985) J. Immunol. Methods 81:31-42; Cote, R. J. et al. (1983) Proc. Natl. Acad. Sci. 80:2026-2030; Cole, S. P. et al. (1984) Mol. Cell Biol. 62:109-120). Briefly, the procedure is as follows: an animal is immunized with the target epitope or immunogenic fragments or conjugates thereof. For example, haptenic oligopeptides of the target epitope can be conjugated to a carrier protein to be used as an immunogen. Lymphoid cells (e.g. splenic lymphocytes) are then obtained from the immunized animal and fused with immortalizing cells (e.g. myeloma or heteromyeloma) to produce hybrid cells. The hybrid cells are screened to identify those that produce the desired antibody.

Human hybridomas that secrete human antibody can be produced by the Kohler and Milstein technique. Although human antibodies are especially preferred for treatment of human, in general, the generation of stable human-human hybridomas for long-term production of human monoclonal antibody can be difficult. Hybridoma production in rodents, especially mouse, is a very well established procedure and thus, stable murine hybridomas provide an unlimited source of antibody of select characteristics. As an alternative to human antibodies, the mouse antibodies can be converted to chimeric murine/human antibodies by genetic engineering techniques. See V. T. Oi et al., Bio Techniques 4(4):214-221 (1986); L. K. Sun et al., Hybridoma 5 (1986).

In addition, techniques developed for the production of “chimeric antibodies”, the splicing of mouse antibody genes to human antibody genes to obtain a molecule with appropriate antigen specificity and biological activity can be used (S. L. Morrison, et al. Proc. Natl. Acad. Sci. 81, 6851-6855 (1984); M. S. Neuberger et al., Nature 312:604-608 (1984); S. Takeda, S. et al., Nature 314:452-454 (1985)). Alternatively, techniques described for the production of single chain antibodies may be adapted, using methods known in the art, to produce target-specific single chain antibodies. Antibodies with related specificity, but of distinct idiotypic composition, may be generated by chain shuffling from random combinatorial immunoglobin libraries (D. R. Burton, Proc. Natl. Acad. Sci. 88, 11120-3 (1991)).

Antibodies may also be produced by inducing in vivo production in the lymphocyte population or by screening immunoglobulin libraries or panels of highly specific binding reagents as disclosed in the literature (R. Orlandi et al., Proc. Natl. Acad. Sci. 86, 3833-3837 (1989)); G. Winter et al., Nature 349, 293-299 (1991)).

### 3. Pharmaceutical Formulations.

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well-known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces such as by aerosol administration as described, for example, in U.S. Pat. No. 4,501,729) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy, which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet could be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations of the present invention suitable for parenteral administration comprise sterile aqueous and non-aqueous injection solutions of the active compound(s), which preparations are preferably isotonic with the blood of the intended recipient. These preparations may contain anti-oxidants, buffers, bacteriostats and solutes, which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s), or a salt thereof, in a unit dosage form in a sealed container. The compound or salt is provided in the form of a lyophilizate, which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent, which is physiologically acceptable may be employed in sufficient quantity to emulsify the compound or salt in an aqueous carrier. One such useful emulsifying agent is phosphatidyl choline.

In addition to active compound(s), the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

The active and supplemental compounds described herein may be administered to the lungs of a patient by any suitable means, but are preferably administered via an aerosol suspension of respirable particles comprised of the active compound, which the subject inhales. The active compound can be aerosolized in a variety of forms, such as, but not limited to, dry powder inhalants, metered dose inhalants, or liquid/liquid suspensions. The respirable particles may be liquid or solid.

The particulate pharmaceutical composition may optionally be combined with a carrier to aid in dispersion or transport. A suitable carrier such as a sugar (i.e., lactose, sucrose, trehalose, mannitol) may be blended with the active compound or compounds in any suitable ratio (e.g., a 1 to 1 ratio by weight).

Solid or liquid particulate forms of the active compound prepared for practicing the present invention should include particles of respirable size: that is, particles of a size sufficiently small to pass through the mouth and larynx upon inhalation and into the bronchi and alveoli of the lungs. In general, particles ranging from about 1 to 10 microns in size are within the respirable range. Particles of non-respirable size that are included in the aerosol tend to be deposited in the throat and swallowed, and the quantity of non-respirable particles in the aerosol is preferably minimized.

In the manufacture of a formulation according to the invention, active compounds of the present invention or the pharmaceutically acceptable salts or free bases thereof are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, which may contain from 0.5% to 99% by weight of the active compound. One or more active compounds may be incorporated in the formulations of the invention, which formulations may be prepared by any of the well-known techniques of pharmacy consisting essentially of admixing the components.

Aerosols of liquid particles comprising the active compound may be produced by any suitable means, such as with a pressure-driven aerosol nebulizer or an ultrasonic nebulizer. See, e.g., U.S. Pat. No. 4,501,729. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier, the active ingredient comprising up to 40% w/w of the formulation, but preferably less than 20% w/w. The carrier is typically water or normal saline or phosphate-buffered saline [PBS] (and most preferably sterile, pyrogen-free water or normal saline or PBS) or a dilute aqueous alcoholic solution, preferably made isotonic but may be hypertonic with body fluids by the addition of, for example, sodium chloride. Optional additives include preservatives if the formulation is not made sterile, for example, methyl hydroxybenzoate, antioxidants, flavoring agents, volatile oils, buffering agents and surfactants.

Aerosols of solid particles comprising the active compound may likewise be produced with any solid particulate medicament aerosol generator. Aerosol generators for administering solid particulate medicaments to a subject produce particles, which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders, which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation. A second type of illustrative aerosol generator comprises a metered dose inhaler. Metered dose inhalers are pressurized aerosol dispensers, typically containing a suspension or solution formulation of the active ingredient in a liquified propellant. During use these devices discharge the formulation through a valve adapted to deliver a metered volume, typically from 10 to 200 μl, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents.

Any propellant may be used in carrying out the present invention, including both chlorofluorocarbon-containing propellants and non-chlorofluorocarbon-containing propellants. Thus, fluorocarbon aerosol propellants that may be employed in carrying out the present invention including fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Examples of such propellants include, but are not limited to, those described in U.S. Pat. No. 6,451,288.

Compositions containing respirable dry particles of micronized active compound of the present invention may be prepared by grinding the dry active compound with, e.g., a mortar and pestle or other appropriate grinding device, and then passing the micronized composition through a 400 mesh screen to break up or separate out large agglomerates.

The aerosol, whether formed from solid or liquid particles, may be produced by the aerosol generator at a rate of from about 10 to 150 liters per minute. Aerosols containing greater amounts of medicament may be administered more rapidly. Typically, each aerosol may be delivered to the patient for a period from about 30 seconds to about 20 minutes, with a delivery period of about one to ten minutes being preferred.

### 4. Subjects and Methods.

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration, or topically to the airway surfaces or lungs of the subject, such as by aerosol inhalation.

Subjects to be treated or tested by the methods of the present invention are those who have been exposed to any level of potentially damaging ionizing radiation. For example, the subjects may be those exposed to 50 or 100 rads; 0.5 or 1 Grey; or 500 to 100 milliSieverts of ionizing radiation, or more. It is generally believed that radiation injury is characterized by delayed onset of symptoms after exposure to the injuring radiation, so it will be understood that treatment may be administered while the injury is at an early or latent stage, as well as during manifest illness.

The subjects may be those afflicted with an acute or local radiation injury. In some embodiments the subjects are those afflicted with a cutaneous radiation injury (CRI), acute radiation syndrome (ARS) or a component thereof (that is, one or more of hematopoietic syndrome, gastrointestinal syndrome, ad cerebrovascular syndrome), radiation injury to the nervous system or brain, radiation pneumonitis, radiation-induced enteritis, etc.

The therapeutically effective dosage of any specific compound, the use of which is in the scope of present invention, will vary somewhat from compound to compound, and patient to patient, and will depend upon the condition of the patient and the route of delivery. In some embodiments, a dosage from about 0.1 to about 50 mg/kg will have therapeutic efficacy, with all weights being calculated based upon the weight of the active compound, including the cases where a salt is employed. In some embodiments, a dosage from about 10 mg/kg to about 50 mg/kg may be employed for oral administration. In some embodiments, a dosage from about 0.5 mg/kg to 5 mg/kg may be employed for intramuscular injection. In some embodiments, a dosage of from about 0.5 or 1 mg/kg up to about 20 mg/kg may be employed for administration to the lungs, such as by aerosol administration.

The present invention is explained in greater detail in the following non-limiting Examples.

### EXAMPLES

Our novel paradigm links GRP to RT lung injury. We hypothesized that GRP is a mediator of RTP: promoting both inflammatory pneumonitis and fibrosis^{6,7}. We propose that ionizing radiation triggers pulmonary neuroendocrine cell (PNEC) hyperplasia with GRP secretion, which then mediates acute and chronic lung injury. GRP receptor gene expression is detected and functional in pulmonary epithelial cells, fibroblasts, endothelial cells, and macrophages⁸⁻¹².

GRP is a proinflammatory neuropeptide that functions as an inflammatory cell activator, mitogen, and cell differentiation factor^{6,8,12}. GRP is present at highest levels in PNEC in fetal lung¹³, in which it can promote development. After birth, GRP production normally drops, but elevated levels are associated with many inflammatory lung conditions, including chronic lung disease of newborns (bronchopulmonary dysplasia, BPD)¹⁴⁻¹⁷. PNEC hyperplasia can be triggered by inflammation or exposure to oxygen^{9,16}, and can take weeks to reach peak levels¹⁸.

Our first investigation tested the hypothesis that GRP contributes to RTP using an established mouse model of radiation fibrosis in C57BL/6 mice, which are prone to fibrosis. Following exposure to high-dose thoracic radiation (15 Gy), we treated mice intraperitoneally (IP) with either PBS or GRP blockade by using small molecule 77427. We quantified results of immunohistochemistry by using quantitative image analysis with ImageJ to determine whether GRP contributes to RT-induced inflammatory responses and/or fibrosis, specifically including assessment of active TGFβsignaling⁷.

In a second series of experiments, C57L/J (L/J) mice were treated with 15 Gy of thoracic radiation. L/J mice are exquisitely sensitive to even low doses of RT, and rapidly develop both pneumonitis and fibrosis. Groups of L/J mice were injected intraperitoneally with PBS, 77427 or 2A11, given as a single dose (20 nmole per mouse) at 24 hours post-RT.

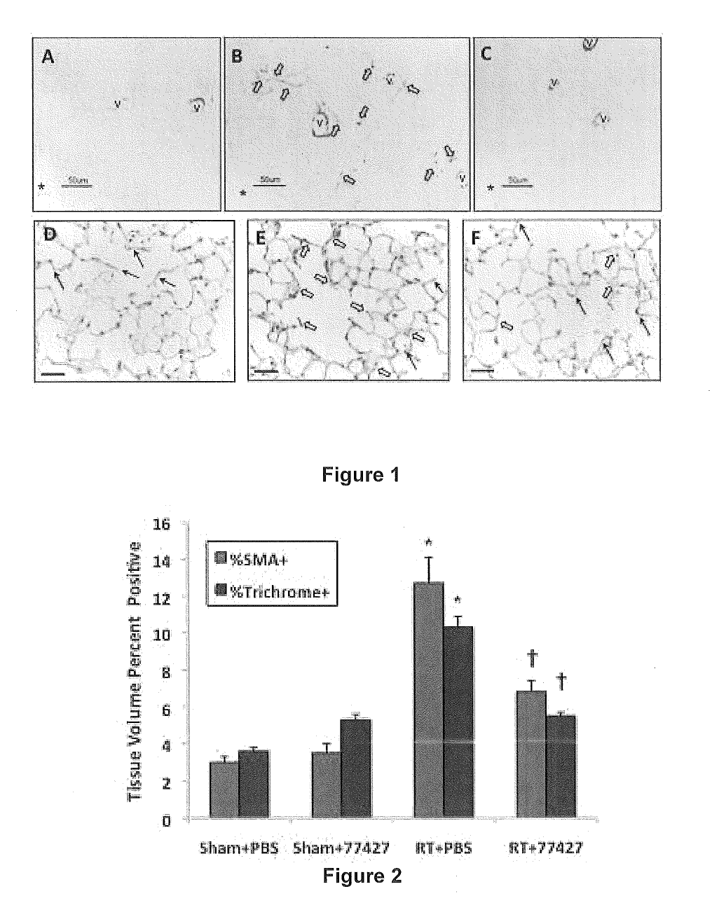

As noted above, the first investigation (FIGS. 1 and 2) tested the hypothesis that GRP contributes to RTP using an established mouse model of radiation fibrosis in C57BL/6 mice. Following exposure to high-dose thoracic radiation (15 Gy), we treated mice intraperitoneally (IP) with either PBS or GRP blockade by using small molecule 77427. We quantified results of immunohistochemistry by using quantitative image analysis with ImageJ to determine whether GRP contributes to RT-induced inflammatory responses and/or fibrosis, specifically including assessment of active TGFβ signaling⁷.

In FIG. 1B, note that there is extensive immunostaining for myofibroblasts in the alveolar walls (arrows) in mice exposed to RT 20 weeks earlier and given only PBS. Treatment with 77427 twice a week for 20 weeks abrogates this interstitial SMA staining, which is an early indicator of radiation fibrosis. Normal vascular-associated SMA staining (“V”) is present in all 3 groups of mice (A,B,C). The pleural surface is indicated by an asterisk (*).

The volume percent of immunostaining for either SMA or Trichrome was normalized for the total alveolar tissue volume, excluding the conducting airways and associated blood vessels. This was carried out by a board-certified anatomic pathologist (M.E.S.) without knowledge of the experimental groups using ImageJ1.62 software. 10-15 random 40× photomicrographs per mouse were captured. Results of this computerized image analysis are given in FIG. 2.

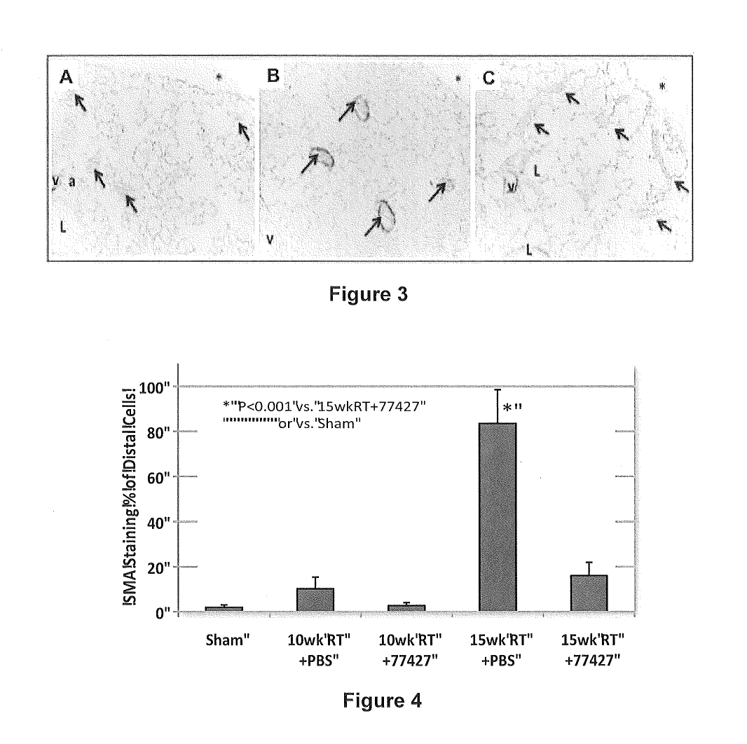

It was recently observed by Zelcko Vujaskovic et al that C57L/J (L/J) mice have radiation sensitivity very similar to humans¹⁹, making the L/J model of radiation-pneumonitis most clinically relevant. Using this L/J model, we treated mice IP with a single dose of 77427 (10 nmol per mouse) given IP at 24 hours post-RT. At 15 weeks later, we observed early evidence of pulmonary fibrosis that was abrogated by the single dose of 77427 (FIG. 3). The localization of the SMA+ cells to small blood vessels indicates that pulmonary vascular pathology can sometimes precede alveolar interstitial fibrosis, suggesting that pulmonary arterial hypertension could either precede or occur independently of pulmonary interstitial fibrosis. Therefore, we have included a claim that GRP blockade may be a treatment to prevent, arrest, or reverse pulmonary arteriopathy leading to pulmonary hypertension, another serious and poorly understood medical condition that is now treated symptomatically.

FIG. 4 shows a quantification of the SMA immunopositive area as shown in FIG. 3 (excluding conducting airways and associated vasculature), normalized for the total area of alveolar tissue.

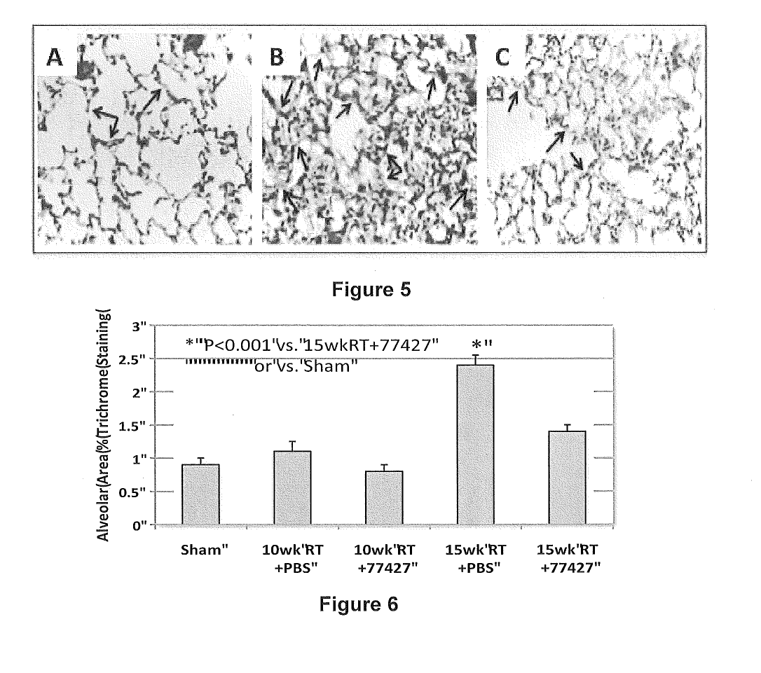

We also analyzed the amount of collagen present in the alveolar interstitium by using the Masson's trichrome staining method for demonstrating collagens in tissue sections²⁰. In normal, Sham-irradiated lung (FIG. 5A), there are only traces of green collagen staining in the alveolar walls (arrows). In contrast, 15 weeks post-RT, early collagen deposition is widespread throughout the alveolar walls. However, treatment with a single dose of 77427 reduced trichrome levels in the alveoli to near baseline levels (FIG. 5C and FIG. 6).

We quantified the area of green trichrome staining in the trichrome-stained slides (including those shown in FIG. 5), using ImageJ, normalized for the total area of alveolar tissue in the same photomicrograph, similar to the analysis used for FIG. 4. L/J lungs assessed at 15 weeks post-RT had about a 2.5-fold increase in the volume percent of alveolar tissue staining normalized for lung area, that was statistically significant compared to either Sham controls or compared to the RT+77427 group (P<0.001), Therefore, GRP blockade abrogates alveolar collagen deposition 15 weeks post-high dose RT.

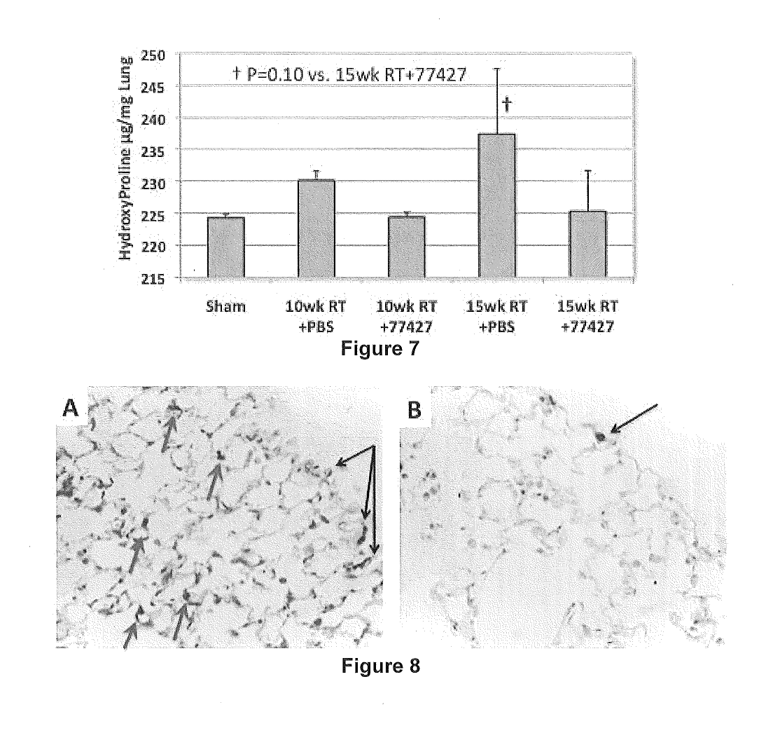

As a second measure of pulmonary collagen levels, we carried out the hydroxyproline assay on whole mouse lungs from the same groups of mice as given in the legend to FIG. 3. Initial assay results are given in FIG. 7. There is a trend toward increased collagen at 15 weeks post-RT, which is confounded to some extent by the presence of collagen in connective tissue of blood vessels and airways in normal lung. Additional samples are being assayed in the near future. In contrast, mice at 15 weeks post-RT+77427 do not have elevated hydroxyproline content.

We became interested in the possibility that increased numbers of pulmonary interstitial mast cells could be one mechanism linking elevated GRP levels to ultimate pulmonary fibrosis. Radiation can induce mast cell degranulation²¹, and leads to increased numbers of pulmonary mast cells^{22,23}. Several mast cell-derived mediators can mediate fibrosis, including tryptase^{21,24} and histamine²⁵. Mast cells can contribute to pulmonary fibrosis in animal models and in patients^{25,26}.

Previously, we demonstrated that GRP promotes mast cell proliferation and chemotaxis (Subramaniam, 2003). Pulmonary interstitial mast cells are greatly increased in lungs of newborn animals treated with 100% oxygen, and this increase is abrogated by GRP blockade (Subramaniam, 2003).

As shown in FIG. 8, we carried out toluidine blue staining for mast cells in lung sections from the same groups of mice given in FIG. 3. There were no detectable subpleural or interstitial mast cells in any sham controls, nor in any lungs at 10 weeks post-RT. At 15 weeks post-RT there was a dramatic accumulation of mast cells subpleurally (black arrows) and in the alveolar interstitium (red arrows). These were most abundant in mice treated with RT+PBS (FIG. 8A), with a reduction by over 50% in mice give 77427 (FIG. 8B). Total numbers of subpleural mast cells have been quantified for all of the lung tissue on each slide and will be normalized for the length of pleura present.

### REFERENCES CITED

1. Vujaskovic, Z., Marks, L. B. & Anscher, M. S. The physical parameters and molecular events associated with radiation-induced lung toxicity, Semin Radiat Oncol 10, 296-307. (2000).
2. Dorr, W., Baumann, M. & Herrmann, T. Radiation-induced lung damage: a challenge for radiation biology, experimental and clinical radiotherapy. Int J Radiat Biol 76, 443-446. (2000).
3. Morgan, G. W. & Breit, S. N. Radiation and the lung: a reevaluation of the mechanisms mediating pulmonary injury. Int J Radiat Oncol Biol Phys 31, 361-369 (1995).
4. Marks, L. B., et al. Radiation-induced lung injury. Semin Radiat Oncol 13, 333-345, (2003).
5. Vujaskovic, Z., et al. A small molecular weight catalytic metalloporphyrin antioxidant with superoxide dismutase (SOD) mimetic properties protects lungs from radiation-induced injury. Free Radic Biol Med 33, 857-863 (2002).
6. Majumdar, I. D, & Weber, H. C. Biology of mammalian bombesin-like peptides and their receptors. Curr Opin Endocrinol Diabetes Obes 18, 68-74 (2010).
7. Zhou, S., et al. Radiation-induced lung injury is mitigated by blockade of gastrin-releasing peptide. Am J Pathol 182, 1248-1254 (2013).
8. Siegfried, J. M., et al. Expression of mRNA for gastrin-releasing peptide receptor by human bronchial epithelial cells. Association with prolonged tobacco exposure and responsiveness to bombesin-like peptides. Am. J. Respir. Crit. Care Med. 156, 358-366 (1997).
9. Subramaniam, M., et al. Bombesin-like peptides modulate alveolarization and angiogenesis in bronchopulmonary dysplasia Am J Respir Crit Care Med 176, 902-912 (2007).
10. Jensen, R. T., Battey, J. F., Spindel, E. R. & Benya, R. V. International Union of Pharmacology. LXVIII. Mammalian bombesin receptors: nomenclature, distribution, pharmacology, signaling, and functions in normal and disease states. Pharmacol Rev 60, 1-42, Epub 2007 Nov. 20-30. (2008).
11. De la Fuente, M., Del Rio, M., Ferrandez, M. D. & Hernanz, A. Modulation of phagocytic function in murine peritoneal macrophages by bombesin, gastrin-releasing peptide and neuromedin C. Immunology 73, 205-211. (1991).
12. Zhou, S., Potts, E. N., Cuttitta, F., Foster, W. M. & Sunday, M. E. Gastrin-releasing peptide blockade as a broad-spectrum anti-inflammatory therapy for asthma. Proc Natl Acad Sci USA 108, 2100-2105 (2011).
13. Wharton, J., et al, Bombesin-like immunoreactivity in the lung. Nature 273, 769-770 (1978).
14. Johnson, J. W. C., Mitzner, W., London, W. T., Palmer, A. E. & Scott, R. Betamethasone and the rhesus fetus: Multisystemic effects. Am. J. Obstet. Gynecol. 133, 677-684 (1979).
15. Meloni, F., et al. Urinary levels of bombesin-related peptides in a population sample from northern Italy: potential role in the pathogenesis of chronic obstructive pulmonary disease. Am J Med Sci 315, 258-265. (1998).
16. Sunday, M. E., Yoder, B. A., Cuttitta, F., Haley, K. J. & Emanuel, R. L. Bombesin-like peptide mediates lung injury in a baboon model of bronchopulmonary dysplasia. J. Clin. Invest. 102, 584-594 (1998).
17. Cullen, A., Van Marter, L. J., Moore, M., Parad, R. & Sunday, M. E. Urine bombesin-like peptide elevation precedes clinical evidence of bronchopulmonary dysplasia. Am. J. Respir. Crit. Care Med. 165, 1093-1097 (2002).
18. Sunday, M. E. & Willett, C. G. Induction and spontaneous regression of intense pulmonary neuroendocrine cell differentiation in a model of preneoplastic lung injury. Cancer Res 52, 2677s-2686s (1992).
19. Jackson, I. L., Vujaskovic, Z. & Down, J. D. Revisiting strain-related differences in radiation sensitivity of the mouse lung: recognizing and avoiding the confounding effects of pleural effusions. Radiat Res 173, 10-20 (2010).
20. Gao, F., et al. Short-Term Treatment with a SOD/Catalase Mimetic, EUK-207, Mitigates Pneumonitis and Fibrosis after Single-Dose Total-Body or Whole-Thoracic Irradiation. Radiat Res 178, 468-480 (2012).
21. Albrecht, M., Muller, K., Kohn, F. M., Meineke, V. & Mayerhofer, A. Ionizing radiation induces degranulation of human mast cells and release of tryptase. Int J Radiat Biol 83, 535-541 (2007).
22. Vergara, J. A., Raymond, U. & Thet, L. A. Changes in lung morphology and cell number in radiation pneumonitis and fibrosis: a quantitative ultrastructural study. Int J Radiat Oncol Biol Phys 13, 723-732. (1987).
23. Pesci, A., Bertorelli, G., Gabrielli, M. & Olivieri, D. Mast cells in fibrotic lung disorders. Chest 103, 989-996 (1993).
24. Ruoss, S. J., Hartmann, T. & Caughey, G. H. Mast cell tryptase is a mitogen for cultured fibroblasts. J Clin Invest 88, 493-499 (1991).
25. Veerappan, A., et al. Mast cells: a pivotal role in pulmonary fibrosis. DNA Cell Biol 32, 206-218 (2013).
26. Cha, S. I., et al. Lung mast cell density defines a subpopulation of patients with idiopathic pulmonary fibrosis. Histopathology 61, 98-106 (2012).
   The foregoing is illustrative of the present invention, and is not to be construed as limiting thereof. The invention is defined by the following claims, with equivalents of the claims to be included therein.

1. A method of treating ionizing radiation injury, pulmonary fibrosis, or pulmonary hypertension in a mammalian subject in need thereof, comprising administering said subject a gastrin-releasing peptide (GRP) blocking agent or inhibitor in a treatment effective amount.

2. The method of claim 1, wherein said radiation injury is caused by alpha radiation, beta radiation, neutron radiation, gamma radiation, or X-radiation.

3. The method of claim 1, wherein said radiation injury is to skin, lung, peripheral nerve, brain, or gastrointestinal tissue.

4. The method of claim 1, wherein said radiation injury is radiation pneumonitis or radiation-induced lung injury (RTP), radiation-induced enteritis, or acute radiation syndrome.

5. The method of claim 1, wherein said administering step is carried out as a single dose of said treatment effective amount.

6. The method of claim 1, wherein said administering step is carried out by parenteral injection.

7. The method of claim 1, wherein said administering step is carried out by topically applying said GRP inhibitor to airway surfaces of said subject.

8. The method of claim 1, wherein said administering step is carried out by inhalation administration.

9. The method of claim 1, wherein said GRP inhibitor is a small molecule GRP inhibitor (e.g., a non-peptide GRP inhibitor).

10. The method of claim 1, wherein said GRP inhibitor is a compound of the formula: 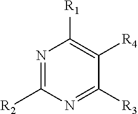
wherein:
R₁ is —R₅—(CH₂)ₙ—CH(R₆)OH, and R₅ is NH, S or O, R₆ is H or CH₃, and n is an integer from 1-4;
R₂ is NH₂, substituted amino or acetamide;
R₃ is H, halogen, CH₃, or CF₃: and
R₄ is H, alkyl, substituted alkyl, alkenyl, alkoxy or halogen;
or a pharmaceutically acceptable salt or prodrug thereof.

11. The method of claim 1, wherein said GRP inhibitor is a compound of the formula: 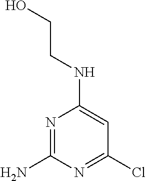 [SMILES: Nc1nc(Cl)cc(NCCO)n1]
or a pharmaceutically acceptable salt or prodrug thereof.

12. The method of claim 1, wherein said GRP inhibitor is the monoclonal antibody 2A11, or a monoclonal antibody that specifically binds to the eptiope bound by the monoclonal antibody 2A11.

13. The method of claim 1, wherein said subject has an elevated level of GRP in at least one tissue or organ at risk of said radiation injury.

14. The method of claim 13, wherein said tissue or organ is lung.

15. The method of claim 1, further comprising the steps of:
determining an elevated level of GRP in a biological sample collected from said subject as compared to normal subjects; and then
administering said GRP inhibitor to said subject when said elevated level is determined.

16. The method of claim 1, wherein said subject is a known airway hyper-reactive subject.

17. The method of claim 1, wherein said ionizing radiation injury is an acute ionizing radiation injury.

18. The method of claim 1, wherein said administering step is carried out within 1, 2 or 3 days of said ionizing radiation injury.

19-20. (canceled)

21. A method of determining if a subject is at increased risk of radiation injury, by determining an elevated level of gastrin-releasing peptide (GRP) in a biological sample collected from said subject as compared to normal subjects, such as urine; normalizing this value to correct for urine concentration, and then classifying said subject as at increased risk of radiation injury if an elevated level of GRP is determined.

22. The method of claim 20, which further comprises at least partially purifying said biological sample.

23. The method of claim 20, which comprises an immunological, chromatographic or electrophoretic determining step.
